(19) United States
(12) Patent Application Publication
(10) Pub. No.: US 2007/0015276 A1
(43) Pub. Date: Jan. 18, 2007

(54) Fluid transfer device

(75) Inventors: Sean J. LeBlanc, Westminster, MA (US); Ian K. MacGregor, Merrimack, NH (US); Bernardo Aumond, Somerville, MA (US); George J. Vella, Medway, MA (US); A. Peter Russo, Woburn, MA (US); Seth T. Rodgers, Somerville, MA (US); Andrey J. Zarur, Woburn, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: BioProcessors Corp., Woburn, MA (US)

(21) Appl. No.: 11/474,050

(22) Filed: Jun. 23, 2006

Related U.S. Application Data
(60) Provisional application No. 60/693,337, filed on Jun. 23, 2005.

(51) Int. Cl. C12M 3/00; A61M 31/00

(52) U.S. Cl. 435/289.1; 604/500

(57) ABSTRACT

Fluid transfer devices described herein can include a body portion and a tip portion. A fluid pathway extends through the body and tip portions through which fluid may be transferred, for example, from a fluid-dispensing apparatus to a fluidic chamber of a microreactor. In some embodiments, the fluid transfer device is connected to the fluid-dispensing apparatus with an engaging element. The engaging element may be part of the body, and can enable the dispensing apparatus to repeatedly engage the body at one predetermined position. The body is capable of storing the fluid received from the dispensing apparatus. The tip portion may be formed of a rigid material (e.g., a metal), and/or may be configured to repeatedly pierce a septum without damaging either the tip or the body. Advantageously, in certain embodiments, the fluid transfer device can controllably transfer small volumes of fluid (e.g., 1 μL) with a high degree of accuracy.

### RELATED APPLICATIONS

This application claims priority under 35 U.S.C. §119(e) to U.S. Provisional Patent Application No. 60/693,337, filed Jun. 23, 2005, and entitled “Fluid Transfer Device”, which is incorporated herein by reference.

### FIELD OF INVENTION

The present invention generally relates to fluid transfer devices and, more specifically, to fluid transfer devices that can pierce a septum.

### BACKGROUND OF INVENTION

A variety of methods are currently available for transferring fluids from one discrete location to another. For example, micropipettes are commonly used for parallel and/or automated delivery of fluids to high-density well plates and to microfluidic systems, which are useful in applications such as immunoassays, chemical assays, and cell culture. Advances in fluid-transfer technology would find application in a number of different fields.

### SUMMARY OF THE INVENTION

Fluid transfer devices and methods associated therewith are provided.

In one aspect of the invention, a device is provided. The device comprises a body comprising a first material, the body defining a fluid pathway therethrough including a first opening at an upper end and a second opening at a lower end of the body, an engaging element associated with the body, comprising a component of a detent mechanism constructed and arranged to engage a complementary detent component of a fluid dispensing apparatus via the first opening such that an outlet of the fluid dispensing apparatus is in fluid communication with the body, the fluid dispensing apparatus being complementary to the body such that the fluid dispensing apparatus can repeatedly engage the body at one predetermined position of the body in relation to the fluid dispensing apparatus, and a tip portion integrally connected to the body and defining a fluid pathway in fluid communication with the second opening of the body that extends through a distal end of the tip portion, wherein the tip portion comprises a metal.

In another aspect of the invention, a device is provided. The device comprises a body comprising a first material, the body defining a fluid pathway therethrough including a first opening at an upper end and a second opening at a lower end of the body, the body constructed and arranged to store a fluid of at least 0.5 mL, an engaging element associated with the body constructed and arranged to engage a fluid dispensing apparatus via the first opening such that an outlet of the fluid dispensing apparatus is in fluid communication with the body, a tip portion integrally connected to the body and defining a fluid pathway in fluid communication with the second opening of the body that extends through a distal end of the tip portion, wherein the tip portion comprises a metal, and wherein the fluid transfer device, when engaged with the fluid dispensing apparatus, controllably dispenses the fluid with an accuracy of at least 90%.

In another aspect of the invention, a device is provided. The device comprises a body portion having a first length and formed of a first material, the body defining a fluid pathway therethrough including a first opening at an upper end and comprising an engaging element constructed and arranged to engage a fluid dispensing apparatus in a predetermined position relative to the dispensing apparatus for receiving a fluid from the dispensing apparatus, and a tip portion constructed and arranged to reproducibly puncture at least five septa not pre-fabricated for facile puncture, comprising an outlet for delivery of a fluid received by the fluid transfer device from the dispensing apparatus, and having a second length and formed of a second material, the tip portion integrally connected to the body and defining a fluid pathway in fluid communication with the body that extends through a distal end of the tip portion, wherein the ratio of the second length to the first length is less than or equal to 1:4.

In another aspect of the invention, a device is provided. The device comprises a body portion defining a fluid pathway therethrough including a first opening at an upper end and comprising an engaging element constructed and arranged to engage a fluid dispensing apparatus in a predetermined position relative to the dispensing apparatus for receiving a fluid from the dispensing apparatus, and a tip portion constructed and arranged to reproducibly puncture at least five septa not pre-fabricated for facile puncture, comprising an outlet for delivery of a fluid received by the fluid transfer device from the dispensing apparatus, the tip portion integrally connected to the body and defining a fluid pathway in fluid communication with the body that extends through a distal end of the tip portion, wherein the fluid transfer device, when engaged with the fluid dispensing apparatus, controllably dispenses a volume of fluid between a range of 50 microliters and 1 milliliter with an accuracy of at least 90%.

In another aspect of the invention, a method is provided. The method comprises providing a microreactor device comprising a container having a volume of less than about 2 ml including a biological or biochemical reactor including a reaction site constructed and arranged to facilitation cell cultivation, puncturing a portion of the microreactor device with a tip portion of a fluid transfer device, the fluid transfer device comprising a body portion defining a fluid pathway therethrough including a first opening at an upper end and comprising an engaging element which engages a fluid dispensing apparatus in a predetermined position relative to the dispensing apparatus for receiving a fluid from the dispensing apparatus, and a tip portion, which punctures a portion of the microreactor device, comprising an outlet for delivery of a fluid received by the fluid transfer device from the dispensing apparatus, the tip portion integrally connected to the body and defining a fluid pathway in fluid communication with the body that extends through a distal end of the tip portion, and transferring fluid from the fluid transfer device into the microreactor device.

In another aspect of the invention, a method is provided. The method comprises providing a microreactor device comprising a container having a volume of less than about 2 ml including a biological or biochemical reactor including a reaction site constructed and arranged to facilitation cell cultivation, puncturing a portion of the microreactor device with a fluid transfer device comprising a pipette tip having a metal puncture tip portion, and transferring fluid from the fluid transfer device into the microreactor device.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A illustrates schematically a fluid transfer device according to one embodiment of the present invention;

FIG. 1B illustrates schematically a fluid transfer device according to another embodiment of the present invention;

FIG. 2 illustrates schematically a close-up view of the fluid transfer device of FIGS. 1A and 1B according to one embodiment of the present invention;

FIG. 3 illustrates schematically a fluid transfer device in connection with a fluid-dispensing apparatus and a fluidic chip according to one embodiment of the present invention.

### DETAILED DESCRIPTION

The present invention includes a fluid transfer device and methods associated with the same. Although a variety of pipette tips are known in the art for precise delivery of very small volumes of fluid, many of those pipette tips are not equipped for puncturing septa, or otherwise creating new openings in previously un-punctured materials. The present invention provides a fluid delivery device which can take the form of a pipette tip, or can be used in essentially any other fluid delivery environment, and which allows for puncture of previously unpunctured septa, and other arrangements involving insertion of the tip of the device into an environment into which fluid is to be delivered.

Fluid transfer devices and methods of the invention can be used in a variety of settings. One such setting, described in more detail below, involves introduction of fluid from the fluid transfer device into a microreactor that includes a reaction site constructed and arranged to facilitate cell cultivation.

Fluid transfer devices of the invention can be used to deliver fluid from the device or draw fluid into the device. That is, they can be used to deliver fluid from the device into another container, or withdraw fluid from that container into the fluid transfer device. Although the primary description below involves transfer of fluid from the device, it is to be understood that in all arrangements fluid can be drawn into the device from another container.

The following documents are incorporated herein by reference, and define a set of examples of microreactors that can be used with fluid delivery devices of the invention, but do not in any way define a limitation on the types of devices with which the fluid transfer device can be used to deliver and/or remove fluid: International Patent Publication No.: WO 2004/016727 (International Patent Application Serial No.: PCT/US03/25956), filed Aug. 19, 2003 and published on Feb. 26, 2004, entitled “Determination and/or Control of Reactor Environmental Conditions”; U.S. Pat. Publication No.: 2004/0132166 (US patent application Ser. No.: 10/664,046), filed Sept. 16, 2003 and published on Jul. 8, 2004, of the same title; U.S. Pat. Publication No.: 2003/0077817 (U.S. patent application Ser. No. 10/119,917), filed Apr. 10, 2002 and published on Apr. 24, 2003, entitled “Microfermentor Device and Cell Based Screening Method,”; U.S. Pat. Publication No. 2004/0058437 (U.S. patent application Ser. No. 10/457,049), filed Jun. 5, 2003 and published on Mar. 25, 2004, entitled “Materials and Reactor Systems having Humidity and Gas Control”.

Fluid transfer devices described herein can include a body portion and a tip portion. A fluid pathway extends through the body and tip portions through which fluid may be transferred, for example, from a fluid-dispensing apparatus to a fluidic chamber of a microreactor. In some embodiments, the fluid transfer device is connected to the fluid-dispensing apparatus with an engaging element. The engaging element may be part of the body, and can enable the dispensing apparatus to repeatedly engage the body at one predetermined position. The body is capable of storing the fluid received from the dispensing apparatus. The tip portion may be formed of a rigid material (e.g., a metal), and/or may be configured to repeatedly pierce a septum without damaging either the tip or the body. Advantageously, in certain embodiments, the fluid transfer device can controllably transfer small volumes of fluid (e.g., 1 μL) with a high degree of accuracy.

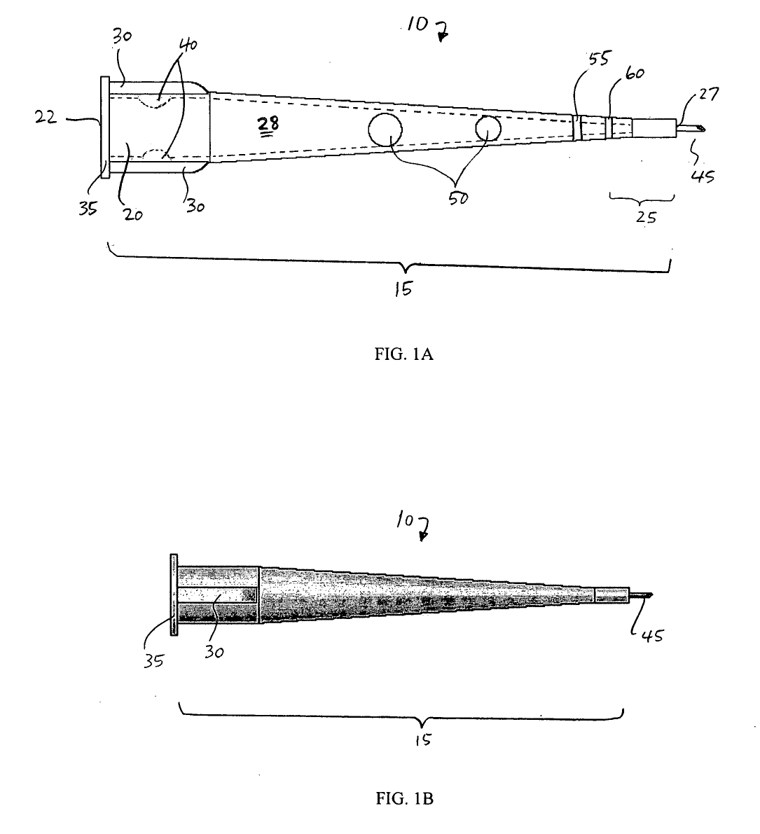

FIGS. 1A and 1B illustrate a fluid transfer device 10 according to one embodiment of the invention. As shown, the fluid transfer device comprises a body 15 having an upper end 20 including a first opening 22, and a lower end 25 including a second opening 27. A fluid pathway 28 is defined within the body between the first and second openings. The body of the fluid transfer device may be used to store a fluid. The first opening can be used to receive a fluid from another source (e.g., from a fluid-dispensing apparatus) and the second opening may be used for transferring a fluid from the body (e.g., into a fluidic channel and/or chamber). Engaging elements 40 may be associated with the body (e.g., near upper end 20), and can be used for connecting the body to an apparatus (e.g., a fluid-dispensing apparatus), described in further detail below. The fluid transfer device may include a tip portion 45 that extends through the second opening at the lower end of the body. In the illustrative embodiment, the body comprises windows 50 that allow optical detection of components of a fluid inside the body. The fluid transfer device may also include a filter 55 positioned within the fluid pathway that separates a component (e.g., a molecule, protein, or cell) from a fluid as the fluid flows from the upper portion to the lower portion of the body. As shown, a mixer 60 may also be incorporated into the fluid transfer device such that a fluid flows turbulently when aspirated out of the tip.

In one embodiment, the first opening of the body has a cross-sectional area that is larger (e.g., between 2-10 times, 10-50 times, or 50-100 times larger) than the cross-sectional area of the second opening 27. The ratio of the cross-sectional areas of the first opening to the second opening may depend, for instance, on the volume of fluid to be stored in the body. A body having a first opening with a cross-sectional area that is 50-100 times larger than that of the second opening may be desirable, for example, when a large volume of fluid (e.g., 5 mL) is stored in the body, so that the fluid can be funneled to the lower portion of the body. Alternatively, if the body is designed to store a smaller amount of fluid (e.g., 1 μL), the cross-sectional area of the first opening may be only 2-10 times larger, or may even have the same cross-sectional area as the second opening.

The length and the width of the body may vary depending on the specific application of the fluid transfer device; for instance, the length and width may depend on the dimensions of the fluid-dispensing apparatus to which the transfer device connects, and/or the amount of fluid to be stored in the body. Generally, the body has a length between 1 to 15 centimeters and a width ranging from a few millimeters to a few centimeters. For instance, the body may have a length of greater than or equal to 2 cm, greater than or equal to 4 cm, greater than or equal to 5 cm, greater than or equal to 7 cm, or greater than or equal to 10 cm.

The body may be designed to store a range of volumes of fluid. For instance, the body may store greater than or equal to 10 μL in some cases; greater than or equal to 50 μL in some cases; greater than or equal to 100 μL in other cases; greater than or equal to 500 μL in yet other cases; greater than or equal to 1 μmL in yet other cases; or, greater than or equal to 5 mL in yet other cases. It may be desirable to store a relatively large volume of fluid in the body (e.g., 5 mL), if, for example, many fluid samples are to be transferred, or if a relatively large amount of fluid (e.g., 100 μL-1 mL) is to be transferred each time. The body may be constructed and arranged to store small amounts of fluid for cases where relatively few samples are to be transferred, or when a relatively small amount of fluid (e.g., 0.1-10 μL) is to be transferred each time.

As shown in FIG. 1A and 1B, the body has a conical shape. It should be understood, however, that the body may have any shape that is suitable for defining a fluid pathway, including a cylindrical, rectangular, square, pyramidal, or irregular shape. In some embodiments, the body may be in the form of a pipette tip.

In one embodiment, the body is comprised of a rigid material so that the fluid transfer device can be used for many fluid transfers without deforming and/or, as discussed below, so that the device can pierce a septum without damaging either the body or the tip. The body may be formed of a material (e.g., a polymer) that is rigid enough to sustain the force needed to pierce through a septum without deforming the body, but is flexible enough to allow for a fluid-tight connection with the fluid-dispensing apparatus. Non-limiting examples of materials that the body can be made of include polymers (such as low density or high density polypropylene, polystyrene, polyethylene, or others), and other materials including glass, quartz, or metals. In some cases, the body comprises a: flexible material such as an elastomer, including silicones (e.g., poly(dimethylsiloxane)) and synthetic rubbers. Those of ordinary skill in the art can readily select a suitable material based upon e.g., its rigidity, its inertness to (i.e., freedom from degradation by) a fluid to be passed through it, its robustness at a temperature at which a particular device is to be used, and/or its transparency/opacity to light (i.e., in the ultraviolet and visible regions).

In some instances, the body is comprised of a combination of two or more materials, such as the ones listed above. For instance, as discussed in more detail below, a fluid transfer device having a body that is formed of a relatively rigid material (e.g., high density polypropylene) may have a section, such as an upper portion, that is made of a flexible material (e.g., a silicone) which forms an engaging element.

The body may include structural supports, such as the ones shown in FIG. 1A and 1B, 30 and 35, which can make the body more resistant to fracture and/or provide support for one or more components (e.g., a window) of the fluid transfer device. As illustrated in the embodiment in FIGS. 1A and 1B, the structural supports are positioned near the upper end and outside of the body. It should be understood, however, that structural supports may be positioned anywhere along the device, including the tip, and may be found inside and/or outside of the body. Structural supports can have any shape or configuration and/or can include any material that is suitable for supporting a structure of the device.

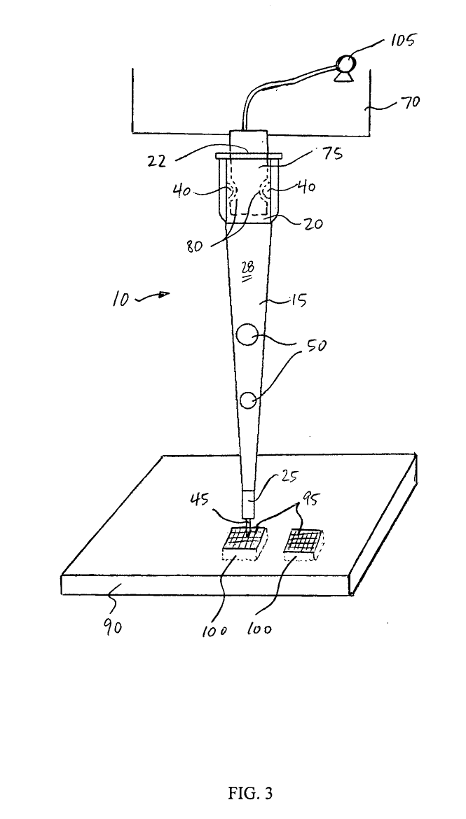

In the illustrative embodiments, the upper end of the body comprises an engaging element 40 that assists in engaging the fluid transfer device to another object, e.g., a fluid-dispensing apparatus. An engaging element may be any appropriate element that connects a fluid transfer device to an object (e.g., a fluid dispensing apparatus) in a suitable manner during use. The engaging element can enable the fluid transfer device to be connected to the fluid-dispensing apparatus in a fluid-tight manner (i.e., without leakage or with minimal evaporation of fluid). The engaging element may comprise different shapes and/or forms. For instance, in some cases, the engaging element has a shape or configuration that is complementary to the attachment end of a fluid-dispensing apparatus, e.g., as shown in FIG. 3. In other cases, the engaging element is deformable such that when a fluid-dispensing apparatus is inserted into the first opening 22 of the body, the engaging element conforms to the shape of the fluid-dispensing apparatus.

The engaging element 40 may comprise a detent mechanism, such as the one shown in FIGS. 1A and 3. A detent mechanism may be any suitable device used to position and/or hold the fluid transfer device in relation to an object (e.g., a fluid dispensing apparatus) so that the fluid transfer device can be released by applying a force to either the transfer device, the object, or both. For instance, the detent mechanism may be in the form of an o-ring, or any other suitable form or configuration. This type of mechanism can allow the fluid-dispensing apparatus 70 to repeatedly engage and secure the body at one predetermined position, e.g., at a predetermined vertical position, as shown in FIG. 3. For example, in a method of use, the fluid-dispensing apparatus 70 may be positioned over the first opening 22 of the body of the fluid transfer device. The fluid-dispensing apparatus can be inserted into the first opening 22, e.g., by being pushed into the internal passageway 28 until the detent mechanism of engaging element 40 is positioned within a complementary detent component, such as a groove 80, of the dispensing apparatus. It should be understood that a complementary detent component may have any shape and/or form suitable for engaging a detent component. The engaging elements and groove cooperate to create a resistance to further movement of the dispensing apparatus relative to the body. This allows the dispensing apparatus and the body to engage in this same position each time they are connected. This is an advantage because the distance between the fluid transfer device and the dispensing location (e.g., a fluidic chamber) can be the same each time without the need to recalibrate the distance between fluid transfers. Vertical control of the fluid transfer device is especially important for chambers having micron-sized dimensions, since it allows the transfer device to transfer fluids, i.e., via puncturing the septa of the chambers, without striking the bottom of the chambers and otherwise damaging the chambers and/or the tip.

In another embodiment, an engaging element can be in the form of a gasket, which may, for example, line a part of the interior portion of the upper end of the body. The engaging element may conform to the shape of the fluid-dispensing apparatus in order to form a liquid-tight seal. The gasket may be formed of an elastomer or any other material suitable for this purpose.

In some cases, the engaging element can function as a locking mechanism, such as one that establishes a mechanical lock between the fluid-dispensing apparatus and the fluid transfer device. Mechanical locks prevent the fluid transfer device from disconnecting from the fluid-dispensing apparatus during normal use. For instance, in one embodiment, a fluid transfer device is rotated in order to disconnect the transfer device from the dispensing apparatus. In another embodiment, a button on a fluid transfer device is pressed while pulling the transfer device away from the dispensing apparatus in order to remove the fluid transfer device. It should be understood that other types and/or configurations of engaging elements than those described herein can be used in combination with the body, including engaging elements that repeatedly engage and secure the body at one predetermined position.

Tip portion 45 allows a fluid from the body to be transferred out of the fluid transfer device. The tip defines a fluid pathway and is in fluid communication with the second opening of the body 27. Fluid flows from the body, through the fluid pathway, and into the tip.

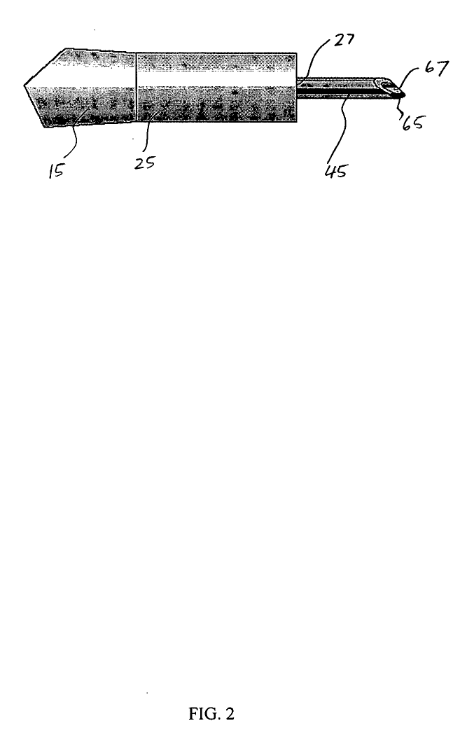

The tip is constructed and arranged to reproducibly puncture a relatively flexible material, such as a septum or a film, and, in some instances, to preserve the fluidic integrity of the material (as discussed in more detail below). The tip may be beveled as to form a pointed portion 65 distal to where the tip joins the body (FIG. 2). The pointed portion of the tip may have a shape such as the one shown in FIG. 2; however, it should be understood that the tip may have any suitable shape that allows a septum or film to be pierced. Advantageously, the septa to be pierced by the tip do not need to be prefabricated for facile puncture, i.e., the septa do not require prefabricated slits or partial openings that aid puncture. For instance, in some embodiments, the tip portion is constructed and arranged to reproducibly puncture at least 5 septa not pre-fabricated for facile puncture, at least 10 septa not pre-fabricated for facile puncture, at least 50 septa not prefabricated for facile puncture, or at least 100 septa not pre-fabricated for facile puncture. In some cases, in order to puncture such septa, the tip portion should be sufficiently sharp. However, in other cases, the tip portion can be blunt (e.g., the tip may not be pointed) depending on, e.g., the method of use and/or the rigidity of the septa.

The tip portion is generally hollow, i.e., defining a fluid pathway 67, as illustrated in FIG. 2. The tip may have any suitable inner diameter for defining the fluid pathway. For instance, in some cases, the tip may be a 21-27 gauge tip (i.e., have a relatively small inner diameter). A tip having a small inner diameter may be advantageous for transferring a small volume of fluid (e.g., 0.1-25 μL), piercing a relatively thin septum (e.g., 10-100 μm thick), making a small puncture in the septum, and/or for generating droplets of fluid having a relatively small diameter (e.g., microns). In other cases, a suitable tip may be a 16-20 gauge tip (i.e., have a relatively large inner diameter). A tip having a large inner diameter may be useful, for example, for transferring larger volumes of fluid (e.g., 1-5 mL), piercing a thicker or more rigid septum, and/or for generating larger droplets of fluid.

The length of the tip portion can range from a few millimeters to centimeters depending on the application or intended use of the fluid transfer device. For instance, the tip may have a length of less than or equal to 3 cm, less than or equal to 1 cm, less than or equal to 6 mm, or less than or equal to 3 mm. In some cases, the ratio of the length of the tip portion to the length of the body is greater than or equal to 1:4, greater than or equal to 1:8, or greater than or equal to 1:16.

A tip having a long length (e.g., 3 cm) can be useful for puncturing several layers of material and/or for transferring fluid to a chamber that is not positioned near the surface of a structure. A tip having a shorter length (e.g., 3 mm) may be desirable, for example, for transferring a fluid into a microfluidic chamber (i.e., a chamber having at least one dimension on the order of 500 microns or less) so that the tip does not puncture or strike the floor of the chamber. A short length can also be advantageous for puncturing a relatively thin septum and/or for minimizing the internal volume of the tip (i.e., for decreasing the dead volume for possible sample carry-over between fluid transfers and/or for increasing the accuracy of the volume transferred).

The tip portion can be made of a rigid material. In some embodiments, the tip portion may be comprised of a metal, such as stainless steel or aluminum. In other embodiments, the tip portion may be comprised of a rigid polymer, glass, fused-silica, or quartz. In some cases, the tip may be formed of a combination of one or materials, such as the ones listed above. Of course, other materials can also be used to form the tip portion. In certain embodiments, the tip portion is formed of a material that is at least as hard, or harder than the material(s) used to form the body. For example, in one embodiment, the body is formed of a first material having a first hardness and the tip portion is formed of a second material having a second hardness, the second hardness being at least 1.5 times that of the first hardness. In another embodiment, the second hardness is at least 2 times that of the first hardness, and in yet another embodiment, the second hardness is at least 5 times that of the first hardness. For instance, the body can be formed of a polymer and the tip portion may be formed of a more rigid polymer, a ceramic, or a metal. Those of ordinary skill in the art can readily select a suitable material for the tip portion based upon, e.g., its rigidity, hardness, and/or its inertness to (i.e., freedom from degradation by) a fluid to be passed through it.

In some instances, all, or a portion, of the tip (e.g., the inner portion defining the fluid pathway and/or the distal end of the tip) may be coated with a hydrophobic or hydrophilic material. Examples of suitable materials include polymers, such as a rubbers, silicones (e.g., poly(dimethylsiloxane)), polytetraflouethylene (PTFE), and others. Coating the tip can change the surface characteristics of the tip, such as the surface tension of the fluid in contact with the tip. The surface characteristics can, in turn, influence the size of a droplet formed at the tip and the volume of fluid transferred.

The tip may be integrally connected to the body. As used herein, the term “integrally connected,” when referring to two or more objects, means objects that do not become separated from each other during the course of normal use, and/or separation requires causing damage to at least one of the components, for example, by breaking, peeling, etc. (separating components fastened together via adhesives, tools, etc.). Various methods of integrally connecting the body and tip portions can be used. For instance, in one embodiment, the tip is integrally connected to the body using an adhesive. In another embodiment, the tip and body are integrally connected by the body being injection molded around the tip. Those of ordinary skill in the art will recognize a variety of techniques for integrally connecting the tip to the body.

In one embodiment, the fluid transfer device includes a filter 55 that separates a component from a fluid as the fluid flows from the upper portion 20 to the lower portion 25 of the body (FIG. 1A). The filter can exclude a component from passing through the distal end of the tip portion and can prevent the component from being transferred when aspirated out of the tip. The filter may be positioned at any point along the body and/or tip and can have any suitable shape or form. For instance, the filter may comprise a membrane, or a porous layer such as a mesh, a gel, a resin, or the like. Different types of filtration, such as filtration based on the size, hydrophobicity/hydrophilicity, and/or charge of a component, may be employed.

A filter that excludes a component based on size (i.e., a size-exclusion filter) may have pores that range in size from nanometers to micrometers. The size of the pores may be chosen depending on the component to be excluded. For instance, a filter having nanometer-sized pores (e.g., 20 nm in diameter) may be suitable for excluding certain viruses from entering the tip portion. Micron-sized pores (e.g., 2 microns) may be appropriate for excluding microorganisms such as bacteria. A filter having pores on the order of˜10-20 microns can separate mammalian cells, and a filter having larger pores (e.g., 20-100 microns) may be suitable for excluding larger components, such as plant cells or insects.

In another embodiment, the fluid transfer device comprises a mixer 60, i.e., features that promote mixing of a fluid in the body. The mixer may be constructed and arranged to make the fluid, in the body flow turbulently when aspirated out of the tip. This may be useful, for example, for mixing the fluid in the body with a component that may be contained inside a reservoir or chamber to which the fluid is transferred.

The mixer may be positioned at any point along the fluid transfer device, including the body and/or the tip portion. The mixer can comprise any suitable shape or form. In some cases, the mixer may comprise baffles or other structures that promote mixing. The mixer may be formed of the same or a different material from the body or tip portion.

Some fluid transfer devices, such as the one shown in FIG. 1A, include one or more transparent windows 50 located along the body of the device. Windows may be used when optically detecting a component of the fluid stored inside the body. Optical detection can be performed directly by a user (e.g., visually by calorimetric detection) or via an instrument (e.g., a spectrophotometer). Detection of a component in a fluid can give an indication of a certain state of the fluid, e.g., if the fluid is suitable for being transferred into a reaction chamber. For instance, detection may include identifying whether or not an enzyme has a certain level of activity suitable for being transferred into a reaction chamber.

As shown in FIG. 1A, the windows are round. However, those of ordinary skill in the art can readily determine a suitable size, shape, and/or position of the window, which may depend on the specific application and/or method of optical detection used. For example, in one embodiment, a body that has two windows that are positioned opposite one another allows transmission measurements of the fluid contained in the space between the two windows. In another embodiment, it may be desirable to have a section of the circumference of the body that is transparent, such that light can pass from one side to the other side of the body without needing to align the body with an optical light source or a detector.

In another embodiment, all, or a portion of, the body may be preconditioned with one or more reagents (e.g., a fluorescently-labeled compound) that is/are capable of reacting with a component of the fluid received from a fluid-dispensing apparatus. For example, a portion of a window may be pre-coated with a chemical compound. A reaction between the two or more components may give rise to an optical signal (e.g., fluorescence) that can be measured through the window.

The one or more windows of the body may be comprised of the same or a different material from the body. For instance, if the window is made of the same material as the body, the window may have thinner walls than the body so as to make the window transparent. The window may be formed of a polymer, including but not-limited to polycarbonate, polyurethane, polyethylene, polystyrene, and poly(dimethylsiloxane). Those of ordinary skill in the art can readily select a suitable material based upon, e.g., its chemical and/or optical properties (e.g., transparency in the ultraviolet and visible ranges), its inertness to (i.e., freedom from degradation by) a fluid to be passed through it, and its robustness at a temperature at which a particular device is to be used.

FIG. 3 shows an example of how fluid transfer device 10 can be used. In operation, a fluid transfer device can transfer fluids into one or many fluidic chips 90, which may be used to perform chemical, biological, or biochemical reactions. A fluid transfer device can be connected manually or robotically to a fluid-dispensing apparatus 70, e.g., by aligning a fluid-dispensing apparatus directly above a fluid transfer device and inserting the former into the latter. The vertical position of the transfer device relative to the fluidic chip can be predetermined by use of an engagement element 40 of the fluid transfer device, which can establish how far the attachment end 75 of the dispensing apparatus is inserted into the passageway 28 of the transfer device as described above. Fluid can be dispensed from the dispensing apparatus into the fluid transfer device, e.g., by using a pumping apparatus 105 connected to the dispensing apparatus. The fluid transfer device can store the volume of fluid received from the dispensing apparatus in the body.

One or several transfer devices (i.e., several devices operated in parallel) can be aligned over a fluidic chip 90, e.g.,a chip that contains one or several ports 95 that are lined with septa. The ports may be fluidically connected to one or more microchambers 100. The tip of the transfer device 45 can be lowered far enough to pierce a septum of the chamber, but not to strike the floor of the port or the chamber. A portion or all of the fluid in each transfer device can be transferred (e.g., in parallel), and the volume and the rate of transfer can be controlled by the fluid-dispensing apparatus. Once a fluid is transferred into the fluidic chip, the fluid transfer device(s) can be aligned with a different chamber or a different chip and this process can be repeated. In some instances, the fluid transfer device is disposable after use.

A wide range of volumes can be transferred from a single fluid transfer device. For instance, in one embodiment, a transfer device having a body that can store up to 1 mL of fluid can dispense a volume ranging from a 1 μL-1 mL. In another embodiment, a fluid transfer device having a storage capacity of 250 μL may transfer a volume ranging from 0.1 μL-250 μL. The increments of volumes transferred may be as small as allowed by the fluid dispensing apparatus and/or the design of the fluid transfer device. In addition, the fluid transfer device can transfer the same or different volumes of fluid during each transfer, i.e., to several fluidic chambers of a chip. The volume dispensed can be controlled, for example, by programming the fluid dispensing apparatus.

In some cases, the fluid transfer device can transfer a volume of fluid with a high degree of accuracy, e.g., with an accuracy of at least 90-95%, at least 95-97%, at least 97-99, or at least 99-99.9%.

The accuracy of the volume transferred by the fluid transfer device may depend on the volume of fluid that can be stored by the transfer device. In some instances, a fluid transfer device that transfers a fluid with 99% accuracy, and that stores 1 mL of fluid, can transfer 1 mL±0.01 mL of fluid. Since the same device can transfer a range of volumes, the device may also transfer 500±5 μL, 250±2.5 μL, 100±1 μL, or 25±0.25 μL of fluid. In other instances, the accuracy of the volume transferred by a fluid transfer device increases as the volume transferred decreases (e.g., for a transfer device that is designed to dispense relatively small volumes of fluid). In other cases, the accuracy of the volume transferred by a fluid transfer device increases as the volume transferred increases (e.g., for a transfer device that is designed to dispense relatively large volumes of fluid).

A range of fluid-dispensing apparatuses may be used with the fluid transfer device described herein. Non-limiting examples include pipettes, microarray pipettors, plungers, fluidic pumps, and the like.

Septa that are pierced by the tip of the fluid transfer device are generally made from flexible materials. Examples of materials that can form septa include, but are not limited to, rubbers, high density polyethylene (“HDPE”), silicones such as poly(dimethylsiloxane) and Formulations RTV 108, RTV 615, or RTV 118 (General Electric, New York, N.Y.), and other polymers such as polyurethane, polytetraflouethylene, and poly(4-methyl-1-pentene) (PMP). In some instances, a septum may be in the form of a membrane or film, i.e., lining one or more ports of a fluidic chip. A septum may be a “self-sealing” material. For instance, when the tip is inserted through a port that is lined with a self-sealing material (e.g., a “self-sealing” port), the material forms a seal generally impermeable to species such as fluids introduced into the chip via the port. A tip can puncture both a septum that is, and a septum that is not, pre-fabricated for facile puncture. A septum that is prefabricated for facile puncture may comprise prefabricated slits and/or partial openings that aid puncture.

Though the fluid transfer device can be used to transfer fluids into a wide variety of reaction sites, chambers, channels, and assay regions, etc., the fluid transfer devices are particularly well-suited for transferring fluids to fluidic chips having small (e.g., micron-sized) dimensions. Examples of suitable fluidic chips include those described in International Patent Publication No.: WO 2004/016727 (International Patent Application Serial No.: PCT/US03/25956), filed Aug. 19, 2003 and published on Feb. 26, 2004, entitled “Determination and/or Control of Reactor Environmental Conditions”; U.S. Patent Publication No.: 2004/0132166 (U.S. patent application Ser. No.: 10/664,046), filed Sep. 16, 2003 and published on Jul. 8, 2004, of the same title; U.S. Patent Publication No.: 2003/0077817 (U.S. patent application Ser. No. 10/119,917), filed Apr. 10, 2002 and published on Apr. 24, 2003, entitled “Microfermentor Device and Cell Based Screening Method,”; U.S. Patent Publication No. 2004/0058437 (U.S. patent application Ser. No. 10/457,049), filed Jun. 5, 2003 and published on Mar. 25, 2004, entitled “Materials and Reactor Systems having Humidity and Gas Control”, which are herein incorporated by reference.

For example, a “reactor” can be a combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to promote and/or monitor a biological; chemical, or biochemical reaction, interaction, operation, or experiment at a reaction site, and which can be part of a chip. For example, a chip may include at least 5, at least 10, at least 20, at least 50, at least 100, at least 500, or at least 1,000 or more reactors. Examples of reactors include chemical or biological reactors and cell culturing devices, as well as the reactors described in International Patent Application Serial No. PCT/US01/07679, published on Sep. 20, 2001 as WO 01/68257, incorporated herein by reference. Reactors can include one or more reaction sites or chambers. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the system in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, At least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a chip. The reaction site may be defined as a region where a reaction is allowed to occur; for example, the reactor may be constructed and arranged to cause a reaction within a channel, one or more chambers, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

In some cases, the reaction site containing cells may include a region containing a gas (e.g., a “gas head space”), for example, if the reaction site is not completely filled with a liquid. The gas head space, in some cases, may be partially separated from the reaction site, through use of a gas-permeable or semi-permeable membrane. In some cases, the gas head space may include various sensors for monitoring temperature, and/or other reaction conditions.

Many embodiments and arrangements are described with reference to a chip, or to a reactor, and those of ordinary skill in the art will recognize that the invention can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

In some embodiments, the reaction site may be defined by geometrical considerations. For example, the reaction site may be defined as a chamber in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site can be very small in certain embodiments. Specifically, the reaction site may have a volume of less than one liter, less than about 100 ml, less than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. The reaction site may have any convenient size and/or shape. In another set of embodiments, the reaction site may have a dimension that is 500 microns deep or less, 200 microns deep or less, or 100 microns deep or less.

In some cases, cells can be present at the reaction site. Sensor(s) associated with the chip or reactor, in certain cases, may be able to determine the number of cells, the density of cells, the status or health of the cell, the cell type, the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site, or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

Chips may also include a plurality of inlets and/or outlets that can receive and/or output any of a variety of reactants, products, and/or fluids, for example, directed towards one or more reactors and/or reaction sites. In some cases, the inlets and/or outlets may allow the aseptic transfer of compounds. At least a portion of the plurality of inlets and/or outlets may be in fluid communication with one or more reaction sites within the chip. In some cases, the inlets and/or outlets may also contain one or more sensors and/or actuators, as further described below. Essentially, the chip may have any number of inlets and/or outlets from one to tens of hundreds that can be in fluid communication with one or more reactors and/or reaction sites. Less than 5 or 10 inlets and/or outlets may be provided to the reactor and/or reaction site(s) for certain reactions, such as biological or biochemical reactions. In some cases each reactor may have around 25 inlets and/or outlets, in other cases around 50 inlets and/or outlets, in still other cases around 75 inlets and/or outlets, or around 100 or more inlets and/or outlets in still other cases.

As one example, the inlets and/or outlets of the chip, directed to one or more reactors and/or reaction sites may include inlets and/or outlets for a fluid such as a gas or a liquid, for example, for a waste stream, a reactant stream, a product stream, an inert stream, etc. In some cases, the chip may be constructed and arranged such that fluids entering or leaving reactors and/or reaction sites do not substantially disturb reactions that may be occurring therein. For example, fluids may enter and/or leave a reaction site without affecting the rate of reaction in a chemical, biochemical, and/or biological reaction occurring within the reaction site, or without disturbing and/or disrupting cells that may be present within the reaction site. Examples of inlet and/or outlet gases may include, but are not limited to, CO₂, CO, oxygen, hydrogen, NO, NO₂, water vapor, nitrogen, ammonia, acetic acid, etc. As another example, an inlet and/or outlet fluid may include liquids and/or other substances contained therein, for example, water, saline, cells, cell culture medium, blood or other bodily fluids, antibodies, pH buffers, solvents, hormones, carbohydrates, nutrients, growth factors, therapeutic agents (or suspected therapeutic agents), antifoaming agents (e.g., to prevent production of foam and bubbles), proteins, antibodies, and the like. The inlet and/or outlet fluid may also include a metabolite in some cases. A “metabolite,” as used herein, is any molecule that can be metabolized by a cell. For example, a metabolite may be or include an energy source such as a carbohydrate or a sugar, for example, glucose, fructose, galactose, starch, corn syrup, and the like. Other example metabolites include hormones, enzymes, proteins, signaling peptides, amino acids, etc.

The inlets and/or outlets may be formed within the chip by any suitable technique known to those of ordinary skill in the art, for example, by holes or apertures that are punched, drilled, molded, milled, etc. within the chip or within a portion of the chip, such as a substrate layer. As described above, in some cases, the inlets and/or outlets may be lined, for example, with an elastomeric material (i.e., to form septa). In certain embodiments, the inlets and/or outlets may be constructed using self-sealing materials that may be re-usable in some cases. For example, an inlet and/or outlet may be constructed out of a material that allows the inlet and/or outlet to be liquid-tight. In some cases, upon puncture of the material using a fluid transfer device and subsequent removal of the fluid transfer device, the material may be able to regain its liquid-tight properties (i.e., a “self-sealing” material).

In some embodiments, the chip may include very small elements, for example, sub-millimeter or microfluidic elements. For example, in some embodiments, the chip may include at least one reaction site having a cross sectional dimension of no greater than, for example, 100 mm, 80 mm, 50 mm, or 10 mm. In some embodiments, the reaction site may have a maximum cross section no greater than, for example, 100 mm, 80 mm, 50 mm, or 10 mm. In other embodiments, a cross section or a maximum cross section of a reaction site may be less than 5 mm, less than 2 mm, less than 1 mm, less than 500 micrometers, less than 300 micrometers, less than 100 micrometers, less than 10 micrometers, or less than 1 micrometer or smaller. As one particular non-limiting example, a reaction site may have a generally rectangular shape, with a length of 80 mm, a width of 10 mm, and a depth of 5 mm.

With regard to throughput, an array of many reactors and/or reaction sites within a chip, or within a plurality of chips, can be built in parallel to generate larger capacities. For example, a plurality of-chips (e.g. at least about 10 chips, at least about 30 chips, at least about 50 chips, at least about 75 chips, at least about 100 chips, at least about 200 chips, at least about 300 chips, at least about 500 chips, at least about 750 chips, or at least about 1,000 chips or more) may be operated in parallel, for example, through the use of robotics, for example which can monitor or control the chips automatically. Additionally, an advantage may be obtained by maintaining production capacity at the small scale of reactions typically performed in the laboratory, with scale-up via parallelization. Many reaction sites may be arranged in parallel within a reactor of a chip and/or within a plurality of chips. Specifically, at least five reaction sites can be constructed to operate in parallel, or in other cases at least about 7, about 10, about 30, about 50, about 100, about 200, about 500, about 1,000, about 5,000, about 10,000, about 50,000, or even about 100,000 or more reaction sites can be constructed to operate in parallel, for example, in a high-throughput system. In some cases, the number of reaction sites may be selected so as to produce a certain quantity of a species or product, or so as to be able to process a certain amount of reactant. In certain cases the parallelization of the chips and/or reactors may allow many compounds to be screened simultaneously, or many different growth conditions and/or cell lines to be tested and/or screened simultaneously. Of course, the exact locations and arrangement of the reaction site(s) within the reactor or chip will be a function of the specific application.

Additionally, any embodiment described herein can be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters. Large volumes may be appropriate where the reactors and/or reaction sites are arranged in parallel within one or more chips, e.g., a plurality of reactors and/or reaction sites may be able to deliver a product to a collection chamber.

In some embodiments, the reaction site(s) and/or the channels in fluidic communication with the reaction site(s) are free of active mixing elements, such as blades, stirrers, or the like, which are movable relative to the reaction site(s) and/or channels themselves, that is, movable relative to portion(s) of the reactor defining a reaction site a or a channel. In these embodiments, the reactor of the chip can be constructed in such a way as to cause turbulence in the fluids provided through the inlets and/or outlets, thereby mixing and/or delivering a mixture of the fluids, preferably without active mixing, where mixing is desired. Specifically, the reactor and/or reaction site(s) may include a plurality of obstructions in the flow path of the fluid that causes fluid flowing through the flow path to mix. These obstructions can be of essentially any geometrical arrangement for example, a series of pillars.

Chips can be constructed and arranged such that they are able to be stacked in a predetermined, pre-aligned relationship with other, similar chips, such that the chips are all oriented in a predetermined way (e.g., all oriented in the same way) when stacked together. When a chip is designed to be stacked with other, similar chips, the chip often can be constructed and arranged such that at least a portion of the chip, such as a reaction site, is in fluidic communication with one or more of the other chips and/or reaction sites within other chips. This arrangement may find use in parallelization of chips.

In one set of embodiments, the chip is constructed and arranged such that the chip is able to be stably connected to a microplate, for example, as defined in the 2002 SPS/ANSI proposed standard (e.g., a microplate having dimensions of roughly 127.76±0.50 mm by 85.48±0.50 mm). As used herein, “stably connected” refers to systems in which two components are connected such that a specific motion or force is necessary to disconnect the two components from each other, i.e., the two components cannot be dislodged by random vibration or displacement (e.g., accidentally lightly bumping a component). The components can be stably connected by way of pegs, screws, snap-fit components, matching sets of indentations and protrusions, or the like. A “microplate” is also sometimes referred to as a “microliter” plate, a “microwell” plate, or other similar terms known to the art. The microplate may include any number of wells. For example, as is typically used commercially, the microplate may be a six-well microplate, a 24-well microplate, a 96-well microplate, a 384-well microplate; or a 1,536-well microplate. The wells may be of any suitable shape, for example, cylindrical or rectangular. The microplate may also have other numbers of wells and/or other well geometries or configurations, for instance, in certain specialized applications.

One or more reaction sites may be positioned in association with a chip such that, when the chip is stably connected to other chips and/or microplates, one or more reaction sites of the chip are positioned or aligned to be in chemical, biological, or biochemical communication with, or chemically, biologically, or biochemically connectable with one or more reaction sites of the other chip(s) and/or one or more wells of the microplate(s). “Alignment,” in this context, can mean complete alignment, such that the entire area of the side of a reaction site adjacent another reaction site or well completely overlaps the other reaction site or well, and vice versa, or at least a portion of the reaction site can overlap at least a portion of an adjacent reaction site or well. “Chemically, biologically, or biochemically connectable” means that the reaction site is in fluid communication with another reaction site or well (i.e., fluid is free to flow from one to the other); or is fluidly connectable to the other site or well (e.g., the two are separated from each other by a wall or other component that can be punctured or ruptured, or a valve in a conduit connecting the two can be opened); or the reaction site and other site or well are arranged such that at least some chemical, biological, or biochemical species can migrate from one to the other, e.g., across a semipermeable membrane. As examples, a chip may have six reaction sites that are constructed and arranged to be aligned with the six wells of a 6-well microplate when the chip is stably connected with the microplate (e.g., positioned on top of the microplate), a chip having 96 reaction sites may be constructed and arranged such that the 96 wells are constructed and arranged to be aligned with the 96 wells of a 96-well microplate when the chip is stably connected with the microplate, etc. Of course, in some cases, the chip may be constructed and arranged such that a single reaction site of the chip is aligned with more than one microplate well and/or more than one other reaction site, and/or such that more than one microplate well and/or more than one other reaction site is aligned with a single reaction site of the chip.

Chips also may be constructed and arranged such that at least one reaction site and/or reactor of the chip is in fluid communication with, and/or chemically, biologically, or biochemically connectable to an apparatus constructed and arranged to address at least one well of a microplate, for example, a fluid transfer device can add species to and/or remove species from wells of microplates, and/or can test species within wells of a microplate. In this arrangement, the fluid transfer device may add and/or remove species to/from a reaction site of a chip, and/or test species at reaction sites. In this embodiment, the reaction sites typically are arranged in alignment with wells of the microplate.

Fluidic chips can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use. In this set of embodiments, the chip is constructed and arranged such that when the chip or reactor is filled with water and the inlets and/or outlets sealed, the chip or reactor has an evaporation rate of less than about 100 microliters per day, less than about 50 microliters per day, or less than about 20 microliters per day. In certain cases, a chip or reactor will exhibit an unmeasurable, non-zero amount of evaporation of water per day. The substantially liquid-tight chip or reactor can have a zero evaporation rate of water in other cases.

It should be understood that chips and reactors may have a wide variety of different configurations. For example, the chip may be formed from a single material, or the chip may contain more than one type of reactor, reservoir and/or agent. In some cases, the chip may contain more than one system able to alter one or more environmental factor(s) within one or more reaction sites within the chip. For example, the chip may contain a sealed reservoir and an upper layer that a non-pH-neutral gas is able to permeate across.

To culture cells and to maintain cell culture (and/or to conduct chemical or biochemical reactions), a reactor a chip may be designed to control gases or humidity therein. In one embodiment, humidity control or maintenance may be provided to the chip in the form of a humidity controller and/or a film, optionally with low water permeability relative to the oxygen permeability. As used herein, a “humidity controller” is a device that allows certain gases, such as oxygen, carbon dioxide, or nitrogen to enter the chip, but inhibits the passage of water vapor into the chip. The humidity controller may allow passage of small amounts of water vapor into the chip, but does not allow as much water vapor to enter the chip as at least one other gas, e.g. those listed above. Examples include, but are not limited to, membranes and thin films (e.g., films having a thickness of less than 2 mm). In some embodiments, the humidity controller may be positioned as, or in, a wall of the chip, such as within a wall of a reactor unit or reaction site. In other embodiments, the humidity controller may be positioned such that it is in fluid communication with one or more reaction sites. In some embodiments, each of the reaction sites in the chip may be adjacent to, and/or in fluid communication with a humidity controller. In some cases, the humidity controller may substantially seal at least a portion of the chip.

In one set of embodiments, the humidity control material may include a membrane or a thin film selected to control the passage of gases and/or water vapor therethrough. In one embodiment, the humidity controller is a membrane or a thin film having a desired permeability to one or more gases. The membrane or thin film may be positioned anywhere in the chip where it is able to affect one or more reaction sites in some fashion. For example, the membrane or thin film may be positioned such that it defines the surface of one or more reaction sites.

In one set of embodiments, the membrane or thin film has a thickness of greater than about 10 micrometers, in some cases greater than about 25 micrometers, in some cases greater than about 50 micrometers, in some cases greater than about 75 micrometers, in some cases greater than about 100 micrometers, or in some cases greater than about 150 micrometers while still allowing sufficient oxygen transport therethrough, for instance, to enable cell culture to occur, as further described herein. In some cases, a membrane or a thin film having a thickness of greater than about 50 micrometers may be particularly useful, for example, during manufacturing of the chip. The membrane may have a thickness of less than 1 or 2 millimeters in some cases.

In some cases, it may be desired to incorporate the humidity control material into a structural aspect of the chip, or to incorporate structural aspects of the chip into the humidity control material. Where the humidity control material is intended to provide or supplement support, or will not itself be otherwise adequately supported, the humidity control material may also include a support layer. A support layer may comprise any material or materials that provides desired support. For example, the support layer may include one of the layers that may otherwise be included in the humidity control material for permeability, such as polydimethylsiloxane or polyfluoroorganic materials, or the support layer may comprise a different material, such as glass (for example, PYREX® glass by Corning Glass of Corning, N.Y.; or indium/tin-coated glass), latex, silicon, or the like. The support layer may be positioned anywhere within the humidity control material, for example, as an outer layer or an intermediate layer, and may be positioned to help protect one or more delicate layers. In some embodiments, the use of a support layer may allow a large portion, or nearly all of a reaction site, reactor, or chip to be constructed of the humidity control material. Preferably, the support layer does not significantly impact the permeability of the humidity control material, or the change in permeability may be accounted for in the design of the humidity control material.

In one set of embodiments; the humidity control material is selected to have a certain permeability and/or a certain permeance. As used herein, the “permeability” of a material is given its ordinary meaning as used in the art, i.e., an intrinsic property that generally describes the ability of a gas to pass through the material. In contrast, as used herein, the “permeance” of a material is the actual rate of gas transport through a sample of a material, i.e., an extrinsic property. The permeance of a sample of material is affected by factors such as the area or thickness of the material, the pressure differential across the material, etc. For example, in FIG. 11, the oxygen permeance of two membranes is shown to be dependent on the membrane's thickness.

A chip, in one set of embodiments, may include a humidity control material (e.g., a membrane or a thin film) having a permeability to oxygen greater than about 3.9×10⁻⁸ cm³/s, and in some cases greater than about 4.3×10⁻⁸ cm³/s, and/or a permeability to water vapor lower than about 1.7×10⁻⁷ cm³/s, and in some cases lower than about 1.0×10⁻⁷ cm³/s. It should be appreciated that, while control of oxygen is used as an example herein, other gases such as nitrogen or carbon dioxide may be controlled instead, at permeabilities as noted above, or a combination of gases may be controlled. It should also be appreciated that while, in the example of cells further described below, the lower limit of oxygen transfer and the upper limit of water vapor transfer may typically be desired to be controlled, in other applications, for example, in a chemical synthesis operation, it may be desired to control other parameters, for example, the upper limit of oxygen transfer and lower limit of water vapor transfer, or the lower and upper limits of other gases such as nitrogen or carbon dioxide.

The humidity control material may be used in a wide variety of reactions and interactions. One example of a reaction is cell culture, for example to maintain a cell culture, to increase the number of available cells or cell types, or to produce a desirable cellular product. In some cases, the humidity control material may allow sufficient oxygen to enter by diffusion therethrough to support cell growth. In certain cases, the humidity control material may also be largely impermeable to microorganisms and other cells, for example to prevent contamination. Preferably, the material has low toxicity.

In embodiments where the invention is used in connection with culturing cells, cell culturing may take place over varying lengths of time, depending on the cells being cultured and other factors known to those of ordinary skill in the art. Thus, the design of the chip and the nature of the humidity control material may be adapted to the culture time. For example, the chip or humidity control material may be designed to allow it to withstand the time needed for the culture and is preferably designed to be able to be reused many times. In various embodiments, cell cultures may be performed in 24 hours, 48 hours, 1 week, 2 weeks, 4 weeks, 6 weeks, 3 months, 1 year, continuously, or any other time required for a specific cell culture.

In some cases, the humidity control material is selected to have a permeability and/or a permeance to one or more gases that corresponds to a range acceptable for culturing certain cells. For example, the humidity control material may have a permeability and/or permeance to oxygen high enough, and/or a permeability and/or permeance to water vapor low enough, to allow cell culturing. Examples of such permeabilities include the above-described permeabilities. Those of skill in the art will be able to identify specific ranges of permeabilities of certain materials appropriate for successfully culturing particular cells and cell lines, as well as larger cellular groups, such as microbial and mammalian cells, tissues, tissue engineering constructs, etc.

Examples of permeability ranges of a humidity control material, for example for use in culturing a broad range of cells, include a permeability to oxygen greater than about 100(cm³_{STP} mm/m² atm day), and a permeability to water vapor less than about 6×10⁻⁶(cm³_{STP} mm/m² atm day). As used therein, “STP” refers to “standard temperature and pressure,” referring to a temperature of 273.15K (0° C.) and a pressure of about 10⁵ Pa (1 atm). In another embodiment, the humidity control material may have a permeability to water that is less than about 100(cm³_{STP} mm/m² atm day) and, in other embodiments, less than about 30(cm³_{STP} mm/m² atm day) or less than about 10(cm³_{STP} mm/m² atm day), and an oxygen permeability of at least about 6×10⁶(cm³_{STP} mm/m² atm day), and in some embodiments, at least about 1×10⁷(cm³_{STP} mm/m² atm day), and in other embodiments greater than about 3×10⁷(cm³_{STP} mm/m² atm day) or 1×10⁸(cm³_{STP} mm/m² atm day). Any combination of oxygen permeability and water vapor permeability listed herein can be used. For microbial cells, an example of a suitable range of oxygen permeability is provided by a membrane having a permeability to oxygen permeability greater than about 1×10³(cm³_{STP} mm/m² atm day) and/or a permeability to water vapor is less than about 6×10⁶(cm³_{STP} mm/m² atm day). For mammalian cells, an example suitable range is provided by a membrane having a permeability to oxygen greater than about 100(cm³_{STP} mm/m² atm day) and a permeability to water vapor lower than about 1×10⁵(cm³_{STP} mm/m² atm day).

For humidity control materials having a permeability to oxygen and water vapor, in certain cases, it is desired that the material have very high oxygen permeability and very low permeability to water vapor. For example, the material may have an oxygen permeability of greater than about 1000(cm³_{STP} micrometer/m² day atm), in some cases greater than about 10,000(cm³_{STP} micrometer/m² day atm), and in some cases greater than about 100,000(cm³_{STP} micrometer/m² day atm), and/or a permeability to water vapor less than about 1000(g micrometer/m² day), in some cases less than about 100(g micrometer/m² day), and in some cases less than about 10(g micrometer/m² day).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit; and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A fluid transfer device, comprising:
a body comprising a first material, the body defining a fluid pathway therethrough including a first opening at an upper end and a second opening at a lower end of the body;
an engaging element associated with the body, comprising a component of a detent mechanism constructed and arranged to engage a complementary detent component of a fluid dispensing apparatus via the first opening such that an outlet of the fluid dispensing apparatus is in fluid communication with the body, the fluid dispensing apparatus being complementary to the body such that the fluid dispensing apparatus can repeatedly engage the body at one predetermined position of the body in relation to the fluid dispensing apparatus; and
a tip portion integrally connected to the body and defining a fluid pathway in fluid communication with the second opening of the body that extends through a distal end of the tip portion, wherein the tip portion comprises a metal.

2. The fluid transfer device as in claim 1, wherein the tip portion is constructed and arranged to reproducibly puncture at least five septa not pre-fabricated for facile puncture.

3. The fluid transfer device as in claim 1, wherein the body is a pipette tip.

4. The fluid transfer device as in claim 1, wherein the length of the body is greater than or equal to 2 cm.

5. The fluid transfer device as in claim 1, wherein the length of the body is greater than or equal to 4 cm.

6. The fluid transfer device as in claim 1, wherein the length of the body is greater than or equal to 5 cm.

7. The fluid transfer device as in claim 1, wherein the length of the body is greater than or equal to 7 cm.

8. The fluid transfer device as in claim 1, wherein the body can store a total volume of greater than or equal to 10 microliters.

9. The fluid transfer device as in claim 1, wherein the body can store a total volume of greater than or equal to 50 microliters.

10. The fluid transfer device as in claim 1, wherein the body can store a total volume of greater than or equal to 100 microliters.

11. The fluid transfer device as in claim 1, wherein the body can store a total volume of greater than or equal to 500 microliters.

12. The fluid transfer device as in claim 1, wherein the first material comprises a polymer.

13. The fluid transfer device as in claim 1, wherein the body further comprises a second material.

14. The fluid transfer device as in claim 13, wherein the second material is an elastomer.

15. The fluid transfer device as in claim 1, further comprising a filter positioned in the fluid transfer device, which excludes a component of a fluid from passing through the distal end of the tip portion.

16. The fluid transfer device as in claim 15, wherein the filter comprises pores having an average diameter of less than or equal to 20 microns.

17. The fluid transfer device as in claim 15, wherein the filter comprises pores having an average diameter of less than or equal to 2 microns.

18. The fluid transfer device as in claim 1, further comprising a mixer.

19. The fluid transfer device as in claim 18, wherein the mixer creates turbulence as fluid is being transferred out of the tip portion.

20. The fluid transfer device as in claim 1, wherein the body further comprises a window transparent in the ultraviolet and visible ranges.

21. The fluid transfer device as in claim 20, wherein the window is coated with one or more reagents capable of reacting with a component of a fluid stored in the body.

22. The fluid transfer device as in claim 1, wherein the detent mechanism comprises an o-ring.

23. The fluid transfer device as in claim 1, wherein the complementary detent component comprises a groove.

24. The fluid transfer device as in claim 1, wherein the engagement element comprises a locking mechanism.

25. The fluid transfer device as in claim 1, wherein the tip portion has a length of less than or equal to 3 mm.

26. The fluid transfer device as in claim 1, wherein the tip portion has a length of less than or equal to 6 mm.

27. The fluid transfer device as in claim 1, wherein the tip portion has a gauge of 21.

28. A fluid transfer device, comprising:
a body comprising a first material, the body defining a fluid pathway therethrough including a first opening at an upper end and a second opening at a lower end of the body, the body constructed and arranged to store a fluid of at least 0.5 mL;
an engaging element associated with the body constructed and arranged to engage a fluid dispensing apparatus via the first opening such that an outlet of the fluid dispensing apparatus is in fluid communication with the body;
a tip portion integrally connected to the body and defining a fluid pathway in fluid communication with the second opening of the body that extends through a distal end of the tip portion, wherein the tip portion comprises a metal; and
wherein the fluid transfer device, when engaged with the fluid dispensing apparatus, controllably dispenses the fluid with an accuracy of at least 90%.

29. The fluid transfer device as in claim 28, wherein the tip portion is constructed and arranged to reproducibly puncture at least five septa not pre-fabricated for facile puncture.

30. The fluid transfer device as in claim 28, further comprising a filter positioned in the fluid transfer device, which excludes a component of a fluid from passing through the distal end of the tip portion.

31. The fluid transfer device as in claim 28, further comprising a mixer.

32. The fluid transfer device as in claim 28, wherein the body further comprises a window transparent in the ultraviolet and visible ranges.

33. The fluid transfer device as in claim 28, wherein the engaging element comprises a detent mechanism constructed and arranged to engage a complementary detent component.

34. The fluid transfer device as in claim 33, wherein the detent mechanism comprises an o-ring.

35. The fluid transfer device as in claim 28, wherein the tip portion has a length of less than or equal to 6 mm.

36. The fluid transfer device as in claim 28, wherein the fluid transfer device, when engaged with the fluid dispensing apparatus, controllably dispenses the fluid with an accuracy of at least 95%.

37. The fluid transfer device as in claim 28, wherein the fluid transfer device, when engaged with the fluid dispensing apparatus, controllably dispenses the fluid with an accuracy of at least 97%.

38. The fluid transfer device as in claim 28, wherein the fluid transfer device, when engaged with the fluid dispensing apparatus, controllably dispenses the fluid with an accuracy of at least 99%.

39. A fluid transfer device, comprising:
a body portion having a first length and formed of a first material, the body defining a fluid pathway therethrough including a first opening at an upper end and comprising an engaging element constructed and arranged to engage a fluid dispensing apparatus in a predetermined position relative to the dispensing apparatus for receiving a fluid from the dispensing apparatus; and
a tip portion constructed and arranged to reproducibly puncture at least five septa not pre-fabricated for facile puncture, comprising an outlet for delivery of a fluid received by the fluid transfer device from the dispensing apparatus, and having a second length and formed of a second material, the tip portion integrally connected to the body and defining a fluid pathway in fluid communication with the body that extends through a distal end of the tip portion, wherein the ratio of the second length to the first length is less than or equal to 1:4.

40. The fluid transfer device as in claim 39, wherein the first material of the body has a first hardness and the second material of the tip portion has a second hardness, the second hardness being at least 1.5 times that of the first hardness.

41. The fluid transfer device as in claim 40, the second hardness being at least 2 times that of the first hardness.

42. The fluid transfer device as in claim 40, the second hardness being at least 5 times that of the first hardness.

43. The fluid transfer device as in claim 39, further comprising a filter positioned in the fluid transfer device, which excludes a component of a fluid from passing through the distal end of the tip portion.

44. The fluid transfer device as in claim 39, further comprising a mixer.

45. The fluid transfer device as in claim 39, wherein the body further comprises a window transparent in the ultraviolet and visible ranges.

46. The fluid transfer device as in claim 39, wherein the engaging element comprises a detent mechanism constructed and arranged to engage a complementary detent component.

47. The fluid transfer device as in claim 46, wherein the detent mechanism comprises an o-ring.

48. The fluid transfer device as in claim 39, wherein the tip portion has a length of less than or equal to 6 mm.

49. A fluid transfer device, comprising:
a body portion defining a fluid pathway therethrough including a first opening at an upper end and comprising an engaging element constructed and arranged to engage a fluid dispensing apparatus in a predetermined position relative to the dispensing apparatus for receiving a fluid from the dispensing apparatus; and
a tip portion constructed and arranged to reproducibly puncture at least five septa not pre-fabricated for facile puncture, comprising an outlet for delivery of a fluid received by the fluid transfer device from the dispensing apparatus, the tip portion integrally connected to the body and defining a fluid pathway in fluid communication with the body that extends through a distal end of the tip portion, wherein the fluid transfer device, when engaged with the fluid dispensing apparatus, controllably dispenses a volume of fluid between a range of 50 microliters and 1 milliliter with an accuracy of at least 90%.

50. The fluid transfer device as in claim 49, further comprising a filter positioned in the fluid transfer device, which excludes a component of a fluid from passing through the distal end of the tip portion.

51. The fluid transfer device as in claim 49, further comprising a mixer.

52. The fluid transfer device as in claim 49, wherein the body further comprises a window transparent in the ultraviolet and visible ranges.

53. The fluid transfer device as in claim 49, wherein the engaging element comprises a detent mechanism constructed and arranged to engage a complementary detent component.

54. The fluid transfer device as in claim 53, wherein the detent mechanism comprises an o-ring.

55. The fluid transfer device as in claim 49, wherein the tip portion has a length of less than or equal to 6 mm.

56. The fluid transfer device as in claim 49, wherein the fluid transfer device, when engaged with the fluid dispensing apparatus, controllably dispenses a volume of fluid between a range of 50 microliters and 1 milliliter with an accuracy of at least 95%.

57. The fluid transfer device as in claim 49, wherein the fluid transfer device, when engaged with the fluid dispensing apparatus, controllably dispenses a volume of fluid between a range of 50 microliters and 1 milliliter with an accuracy of at least 97%.

58. The fluid transfer device as in claim 49, wherein the fluid transfer device, when engaged with the fluid dispensing apparatus, controllably dispenses a volume of fluid between a range of 50 microliters and 1 milliliter with an accuracy of at least 99%.

59. A method of transferring a fluid from a fluid transfer device to a microreactor device, comprising:
providing a microreactor device comprising a container having a volume of less than about 2 ml including a biological or biochemical reactor including a reaction site constructed and arranged to facilitation cell cultivation;
puncturing a portion of the microreactor device with a tip portion of a fluid transfer device, the fluid transfer device comprising a body portion defining a fluid pathway therethrough including a first opening at an upper end and comprising an engaging element which engages a fluid dispensing apparatus in a predetermined position relative to the dispensing apparatus for receiving a fluid from the dispensing apparatus, and a tip portion, which punctures a portion of the microreactor device, comprising an outlet for delivery of a fluid received by the fluid transfer device from the dispensing apparatus, the tip portion integrally connected to the body and defining a fluid pathway in fluid communication with the body that extends through a distal end of the tip portion; and
transferring fluid from the fluid transfer device into the microreactor device.

60. The method as in claim 59, wherein the microreactor device includes at least a first microfluidic channel fluidly connecting the first container with an inlet port, the method comprising puncturing a portion of the microreactor device proximate the inlet port thereby transferring fluid from the fluid transfer device through the microfluidic channel and into the first container.

61. A method of transferring a fluid from a fluid transfer device to a microreactor device, comprising:
providing a microreactor device comprising a container having a volume of less than about 2 ml including a biological or biochemical reactor including a reaction site constructed and arranged to facilitation cell cultivation;
puncturing a portion of the microreactor device with a fluid transfer device comprising a pipette tip having a metal puncture tip portion; and
transferring fluid from the fluid transfer device into the microreactor device.
